(19) United States
(12) Patent Application Publication
(10) Pub. No.: US 2014/0277226 A1
(43) Pub. Date: Sep. 18, 2014

(54) EXTERNALLY-SECURED MEDICAL DEVICE

(71) Applicant: PACESETTER, INC., Sylmar, CA (US)

(72) Inventors: John W. Poore, South Pasadena, CA (US); Heather Hamilton, Santa Monica, CA (US); Karl Pawlik, Simi Valley, CA (US); Gene A. Bornzin, Simi Valley, CA (US); George L. Walls, Valencia, CA (US)

(73) Assignee: PACESETTER, INC., Sylmar, CA (US)

(21) Appl. No.: 13/801,624

(22) Filed: Mar. 13, 2013

(51) Int. Cl. A61N 1/39

(52) U.S. Cl. CPC A61N 1/3987
USPC 607/7

(57) ABSTRACT

Certain embodiments of the present disclosure provide an externally-secured medical device (ESMD) configured to be securely affixed to skin of a patient. The ESMD may include at least one pad configured to be directly secured to the skin of the patient. The pad(s) may include at least one electrode configured to direct therapeutic energy into the skin of the patient toward an internal organ. An adhesion component is provided on a patient-engaging surface of the at least one pad configured to securely affix the at least one pad in a persistent and enduring manner to the skin of the patient. The at least one pad is directly secured to the skin of the patient through the adhesion component.

### BACKGROUND

Embodiments of the present disclosure generally relate to an externally-secured medical device configured to be secured directly to an individual, and, more particularly, to a self-contained medical device configured to be worn by an individual.

Numerous medical devices exist today, including but not limited to electrocardiographs (“ECGs”), electroencephalographs (“EEGs”), squid magnetometers, implantable pacemakers, implantable cardioverter-defibrillators (“ICDs”), neurostimulators, electrophysiology (“EP”) mapping and radio frequency (“RF”) ablation systems, and the like. Medical devices commonly employ one or more leads with electrodes that either receive or deliver voltage, current or other electromagnetic pulses (generally “energy”) from or to an organ or tissue (collectively hereafter “tissue”) for diagnostic or therapeutic purposes.

Often, medical devices may be implanted within patient anatomy. However, in order to implant the medical device, a patient typically undergoes surgery. As with any surgery, a risk of complications and adverse consequences exist.

While many medical devices are configured to be implanted within patient anatomy, other medical devices are configured to be external to patient anatomy. For example, a conventional external defibrillator includes defibrillator pads configured to be placed by another individual on a chest of a patient. The defibrillator pads are operatively connected to a large, bulky defibrillating device that is configured to control operation of the defibrillator pads.

In general, an external defibrillator system is used to defibrillate a heart after ventricular arrhythmia has commenced. Typically, the defibrillator pads are applied to a patient by another individual, such as medical personnel.

There are many situations in which patients may be at risk of sudden cardiac death. Often these patients are treated by implanting an implantable defibrillator (ICD). However some patients do not meet the clinical indications for an ICD. Patients that have had an acute MI and an ejection fraction of <35% are not indicated for an ICD until a 40 day waiting period has past. Patients that are on a heart transplant list are not given ICDs. Patients that have newly diagnosed non-ischemic cardiomyopathy with a ejection fraction of <35% must wait 3 to 9 months and be given a full heart failure drug regimen before receiving an ICD. Before and after coronary artery bypass surgery or percutaneous coronary angioplasty there is a 90 day waiting period to see if the patient remains eligible for an ICD. Other ICD implant exclusions include patients with a terminal illness that is not expected to live more than a year or if the patient has NYHA class IV heart failure.

There are alternatives that may be used to protect these patients from cardiac sudden death as a bridge until when they meet the indications for an ICD or until they receive a heart transplant or succumb to terminal illness or cardiac pump failure.

One alternative is to provide the patients care giver with an AED (automatic external defibrillator) that can be administered to the patient by the care giver in the advent of a cardiac arrhythmia. Another alternative is for the patient to wear a defibrillator vest. The vest is intended to be worn continuously except when bathing. Electrodes sense the cardiac electrogram while a separate unit that is electrically attached to the vest monitors the sensed electrogram for an indication of a cardiac arrhythmia. If an arrhythmia commences and the patient is deemed unconscious, a conductive electrode gel is released by the vest to improve electrode coupling to the patients. Then a shock is applied to convert the arrhythmia.

An AED has limitations because the care giver must constantly be vigilant and may miss the episode if not present at all times. Furthermore, it takes some time to apply the electrodes to the patient and thus prolongs the time to therapy administration which has the effect of decreasing the probability of survival.

Further, a defibrillator vest is sometimes deemed uncomfortable when worn around the clock and may not be worn while bathing or in the shower. This puts the patient at risk because it limits compliance.

### SUMMARY

Certain embodiments of the present disclosure provide an externally-secured medical device (ESMD) configured to be securely affixed to skin of a patient. The ESMD may include a sensing module configured to sense cardiac activity and detect arrhythmia, and at least one pad configured to be directly secured to the skin of the patient. The pad(s) may include at least one charge storage element operatively connected to at least one switch, a battery configured to provide power to the ESMD and to charge the at least one charge storage element, at least one electrode configured to deliver stimulation therapy to the skin of the patient along at least one vector through cardiac tissue, and at least one control unit operatively connected to the at least one charge storage element, the switch, the battery, and the at least one electrode. The control unit is configured to control operation of the ESMD to deliver the stimulation therapy when the arrhythmia is detected. An adhesion component is provided on a patient-engaging surface of the at least one pad. The adhesion component is configured to securely affix the pad(s) in a persistent and enduring manner to the skin of the patient. The pad(s) and the electrode(s) are configured to be directly secured to the skin of the patient through the adhesion component.

The pad(s) may include first and second pads connected together through a coupling member. The electrode(s) may include a first electrode on the first pad, and a second electrode on the second pad. The charge storage element(s) may include at least one capacitor operatively connected to the at least one electrode through the switch. The capacitor(s) may include one or both of a dielectric capacitor or an electrochemical capacitor. The capacitor(s) may include at least one flexible capacitor. The control unit may be formed on a flexible circuit. A memory may be operatively connected to the control unit.

The ESMD may also include a telemetry circuit configured to communicate with an external device. The ESMD may also include a speaker configured to emit audible alerts.

The battery may include a flexible substrate including flexible sheets of polydimethylsiloxane (PDMS). The battery may include a lithium cobalt oxide cathode, a solid electrolyte, and a lithium-metal anode supported by the flexible substrate. The ESMD may include a plurality of electrodes at different orientations.

The pad(s) may include three or more pads connected together through a plurality of coupling members. Additionally, the pad(s) may include positioning indicia configured to allow a clinician to properly position the at least one pad on the skin of the patient.

Certain embodiments of the present disclosure provide a method of securing an externally-secured medical device (ESMD) to skin of a patient. The method may include applying an adhesion component to at least one pad of the ESMD, determining a position of the at least one pad on the skin of the patient, and pressing the at least one pad into the skin so that the adhesion component secures the at least one pad to the skin. The pressing operation may include positioning the at least one pad so that at least one electrode is configured to deliver stimulation therapy to the skin of the patient along at least one vector through cardiac tissue. The pressing operation securely affixes the at least one pad to the skin of the patient in a persistent and enduring manner to the skin of the patient.

The method may also include detecting a baseline ECG after the pressing operation, and before detection of an arrhythmia. The baseline ECG may be used determine or detect the arrhythmia.

### BRIEF DESCRIPTION OF THE DRAWINGS

FIG. 1 illustrates an isometric view of an externally-secured medical device secured proximate to a chest of a patient, according to an embodiment of the present disclosure.

FIG. 1a illustrates a simplified cross-sectional view of a pad, according to an embodiment of the present disclosure.

FIG. 1 b illustrates a simplified cross-section view of a pad, according to an embodiment of the present disclosure.

FIG. 1c illustrates a simplified cross-section view of a pad, according to an embodiment of the present disclosure.

FIG. 1d illustrates a bottom view of a pad, according to an embodiment of the present disclosure.

FIG. 1e illustrates a simplified view of first and second pads of an externally-secured medical device in relation to a heart, according to an embodiment of the present disclosure.

FIG. 2 illustrates a simplified schematic diagram of an externally-secured medical device, according to an embodiment of the present disclosure.

FIG. 3a illustrates a simplified schematic diagram of first and second electrodes connected to a charge storage element, according to an embodiment of the present disclosure.

FIG. 3b illustrates a simplified configuration for a charge storage element, according to an embodiment of the present disclosure.

FIG. 4 illustrates a simplified schematic diagram of an externally-secured medical device, according to an embodiment of the present disclosure.

FIG. 5 illustrates a simplified schematic diagram of an externally-secured medical device, according to an embodiment of the present disclosure.

FIG. 6 illustrates a simplified schematic diagram of an externally-secured medical device, according to an embodiment of the present disclosure.

FIG. 7 illustrates a simplified schematic diagram of an externally-secured medical device, according to an embodiment of the present disclosure.

FIG. 8 illustrates a top view of a pad of an externally-secured medical device, according to an embodiment of the present disclosure.

FIG. 9 illustrates an exemplary block diagram of the electrical components of an externally-secured medical device, according to an embodiment of the present disclosure.

FIG. 10 illustrates a functional block diagram of an external device xxx00 that is operated in accordance with the processes described herein and to interface with externally-secured medical devices, according to an embodiment of the present disclosure.

FIG. 11 illustrates a flow chart of a process of securing an externally-secured medical device to a patient, according to an embodiment of the present disclosure.

### DETAILED DESCRIPTION

FIG. 1 illustrates an isometric view of an externally-secured medical device (ESMD) 10 secured proximate to a chest 12 of a patient 14, according to an embodiment of the present disclosure. The ESMD 10 may be one or more of an externally-secured (for example, secured directly to an outer surface of the skin of a patient) and worn ECG, EEG, squid magnetometer, pacemaker, cardioverter, defibrillator, neurostimulator, or the like. For example, the ESMD 10 may be a biphasic defibrillator. The ESMD 10 may include a first pad 16 connected to a second pad 18 through a coupling member 20. The ESMD 10, including the first and second pads 16 and 18, is secured to the patient 14, such as through an adhesive component, such as a medical adhesive, even when the patient 14 is not having an arrhythmia or other adverse cardiac episode. The ESMD 10 is chronically present with respect to the patient 14 in that the ESMD 10 is adhesively secured to the patient 14 over long periods, such as days, weeks, and months, whether the patient 14 experiences adverse cardiac episodes or not. The ESMD 10 is configured to be adhesively secured to skin of the patient 14 through one or more adhesion components provided on patient-engaging surfaces of the ESMD 10, such as patient-engaging surfaces of the first and second pads 16 and 18. The adhesion component(s) is configured to securely affix the ESMD 10 in a persistent and enduring manner to the skin of the patient. That is, the ESMD 10 is securely affixed to the patient over relatively long periods of time, such as days, weeks, and months, such that the ESMD 10 is secured to the patient during periods of normal cardiac activity, and adverse cardiac activity, such as arrhythmias. In other words, the ESMD 10 is adhesively secured to the patient 14 when the patient experiences no or any life-threatening arrhythmias, and continually monitors the cardiac function of the patient 14 to detect adverse cardiac events. If any cardiac events are detected when the ESMD 10 is adhesively secured to the patient 14, the ESMD 10 provides therapeutic energy into the heart in order to return the heart back to normal cardiac activity.

Each of the first and second pads 16 and 18 may include a flexible securing strip 22 integrally connected to a housing 24 that may contain a control unit, electrodes, and/or the like, as discussed below. The flexible securing strip 22 may be integrally connected to the housing 24 in that the flexible securing strip 22 and the housing 24 are contained within a common covering shroud, case, coating, or the like, such as may be formed of a flexible material, such as plastic, elastomeric materials, and the like, that provide flexibility and resilience to the first and second pads 16 and 18. The securing strips 22 are configured to position one or more electrodes (not shown in FIG. 1) in relation to the skin so that the electrode(s) may deliver electrical energy toward and into the heart. Each securing strip 22 may be formed of a flexible plastic that allows the securing strip 22 to move and flex with the contours of the chest 12 and skin of the patient 14. The securing strips 22 may be secured to the skin of the patient through adhesive components, such as medical adhesives that are configured to secure the pads 16 and 18 directly to the skin, while also being conductive and allowing charge to pass therethrough. The securing strips 22 adhere and remain adhesively secured to the skin of the patient 14. For example, the medical adhesives may include double-sided medical tape, adhesive spray, conductive epoxy, latex, glue, or the like that are configured to securely and resiliently secure the pads 16 and 18 directly to outer surfaces of the skin of the patient 14.

U.S. Pat. No. 5,800,685 discloses a highly adhesive material that sticks readily to the skin and affords enough hydration to provide a conductive interface for defibrillation. The conductive adhesive hydrogel comprises cationic acrylates that are synthesized by free radical polymerization in an aqueous environment.

Additives may be added to enhance performance including buffers, humectants, platicizers, pharmaceuticals, and conductivity enhancers. When a conductive screen, or foil is provided underneath such a conductive adhesive, the securing strips 22 also act to secure the pads 16 and 18, as well as forming a portion of the electrode system for delivering electrical current to the patient. The securing strips 22 are configured to be directly secured to the skin through the adhesive. That is, securing strips 22 may directly abut into the skin with only the conductive adhesive between an underside of the securing strips 22 and an outer surface of the skin.

One of the pads 16 or 18 may be configured to be positioned to the right of the mid-sternal area, along adjacent ribs, such as second or third ribs. The other of the pads 16 or 18 may be configured to be positioned mid-axillary, around the fifth rib. However, the pads 16 and 18 may be positioned with respect to various other areas of patient anatomy. The patient-engaging area of each pad 16 and 18 may be 7 cm×10 cm, for example, although smaller or larger pads 16 and 18 may be used. Typically the area of the electrode is at least 50 square cm and the total area of two coupling electrodes may be about 150 square cm. Electrodes having too large of an area may result in a current flux density that is too low and too small of an area may result in too high of an impedance such that too little current is delivered at a given voltage.

In order to secure the pads 16 and 18 to the skin of the patient, the skin may first be slightly abraded. The slight abrasion may allow for more effective adhesion with a medical adhesive.

FIG. 1a illustrates a simplified cross-sectional view of the first pad 16, according to an embodiment of the present disclosure. While the first pad 16 is shown, the second pad 18 may be similarly configured. The first pad 16 includes the securing strip 22 connected to the housing 24. The securing strip 22 and the housing 24 may be contained within a common encapsulating shroud, as noted above. The securing strip 22 represents a flexible substrate that is configured to flex and move in conformity with movement of the patient's skin to which the first pad 16 is adhesively secured. One or more electrodes 25 may be provided on a patient engaging surface 23 and extend downwardly (as shown in FIG. 1a) from the securing strip 22 and may be surrounded by conductive medical adhesive 27 that is configured to contact the patient's skin and provide an adhesively securing medium between the patient's skin and the securing strip 22. The housing 24 may include various electronics, such as capacitors, switches, a control unit, and the like, that are operatively connected to the one or more electrodes 25. Depending on the area of pad 16, the electrodes 25 may be made of a body conformable screen or foil such as aluminum and may extend to the perimeter of the patient engaging surface 23. A conformable conductive adhesive may then be used to cover the electrode 25 to the perimeter of the patient engaging surface 23.

FIG. 1 b illustrates a simplified cross-section view of the first pad 16, according to an embodiment of the present disclosure. While the first pad 16 is shown, the second pad 18 may be similarly configured. In this embodiment, the electrodes 25 are surrounded by conductive adhesive 29. Before application to a patient, the electrodes 25 and adhesive 27 may be covered by a peelable cover 31, such as formed of liquid impermeable plastic, or the like. In the fully-covered position, the cover 31 prevents the adhesive 27 from passing therethrough. When the first pad 16 is to be applied to a patient, the cover 31 is peeled off, as shown in FIG. 1 b, to expose the adhesive 27, at which point the first pad 16 may be adhesively secured to the patient.

The battery, capacitors, control unit, speaker, telemetry unit, and connecting conductor could comprise one unitary structure that could snap into a disposable, renewable adhesive paddle having a snap in docking area and connector for the electronics and include a foil electrode, with conductive adhesive. Thus the system could be worn for a week and the electrode paddles could be replaced while using the same electronics.

FIG. 1c illustrates a simplified cross-section view of a pad 16′, according to an embodiment of the present disclosure. The pad 16′ may be used in place of any of the pads described in the present application. While the first pad 16′ is shown, a second pad 18′ may be similarly configured. In this embodiment, a patient engaging surface 23′ may include a single electrode 25′. For example, instead of multiple electrodes, most, if not all, of the patient engaging surface 23′ may be defined by the unitary electrode 25′, which may be surrounded and covered by a conductive adhesive, as described above. The electrode 25′ may be made of flexible foil or screen and a metal such as aluminum which is flexible, highly conductive and relatively low cost.

FIG. 1d illustrates a bottom view of the pad 16′, according to an embodiment of the present disclosure. As shown in FIG. 1d, the electrode 25′ may be covered by a conductive metal foil screen 29′ that is configured to contact skin of an individual. The electrode 25′ may occupy most, if not all, of the bottom surface area of the pad, with the exception of an outer frame or rim 31′. Furthermore, the electrode 25′ may be coated with a conductive adhesive. Alternatively, the patient engaging surface 23′ may be made of a conductive gel while the outer frame 31′ may be coated with an adhesive that may or may not be conductive.

Referring again to FIG. 1, the coupling member 20 may include a flexible, insulated wire that mechanically and electronically connects the pads 16 and 18 together. The coupling member 20 may be formed of an elastomeric material, flexible plastic, or the like that is flexible, resilient, and water-proof. The coupling member 20 may route internal communication, power, and therapy delivery wires between the housings 24 of the first and second pads 16 and 18. Additionally, medical adhesive may be applied to an underside of the coupling member 20 to adhesively secure the coupling member 20 to the skin of the patient 14.

As an alternative, the coupling member 20 may be separable from the first and second pads 16 and 18 respectively. In this embodiment, the pads 16 and 18 may be first applied to the skin of the patient, as discussed above. Once the pads 16 and 18 are properly positioned, the coupling member 20 may be connected to each of the first and second pads 16 and 18, thereby securing the pads 16 and 18 together. For example, each of the first and second pads 16 and 18 may include electrical ports configured to receive terminal ends of the coupling member 20. The terminal ends of the coupling member 20 may be configured to be removably retained within the respective ports, such as through snapable, latchable, or the like interfaces.

In operation, the ESMD 10 may be configured for external defibrillation. As such, the ESMD 10 may be configured to deliver one or more therapeutic pulses of electrical energy to the heart. The electrical energy may pass from the pads 16 and 18, through the conductive adhesive, and into an internal organ, such as the heart, through the skin and other intervening anatomy. During defibrillation, for example, electrical energy delivered to the heart from electrodes within the pads 16 and 18 depolarizes a critical mass of the heart muscle, and allows normal sinus rhythm to be re-established.

As shown in FIG. 1, the ESMD 10 may be in an anterior-apex orientation. In the anterior-apex orientation, the first pad 16 is placed to the right of the heart, below the clavicle, while the second pad 18 is applied to the left of the heart, just below and to the left of the pectoral muscle. However, the ESMD 10 may be configured for use in various other orientations. For example, the ESMD 10 may be positioned in an anterior-posterior orientation, in which the first pad 16 is placed over the left precordium (for example, over the lower part of the chest, in front of the heart), while the second pad 18 is placed on the back, behind the heart. In such an embodiment, the coupling member 20 may be longer than shown in FIG. 1. The ESMD 10 may include more pads 16 and 18 than shown. For example, the ESMD 10 may include three, four, or more pads, each having one or more electrodes. Additional electrodes allow for additional sensing and therapy vectors. For example, a first electrode may be directed toward a superior vena cava, a second electrode may be directed toward a ventricular vestibule, a third electrode may be directed toward an apex of the heart, while a fourth electrode may be directed toward an inferior vena cava, for example. Each combination of electrodes provides a separate and distinct therapy vector.

FIG. 1e illustrates a simplified view of first and second pads 16 and 18 of an ESMD 10 in relation to a heart 200, according to an embodiment of the present disclosure. The dashed lines showing the first and second pads 16 and 18 show alternative arrangements for the first and second pads 16 and 18. Embodiments are described herein, whereby multiple active electrodes within the first and second pads 16 and 18 are utilized to provide therapy to the heart 200 along therapy vectors.

The ESMD 10 defines therapy vectors between various combinations of two or more electrodes within the first and second pads 16 and 18. The therapy vectors represent paths (generally a linear path) between at least two points.

For example, the therapy vector 202 extends between one or more electrodes within the first pad 16 and one or more electrodes in the second pad 18. As such, the therapy vector 202 may pass from the first pad 16 through the right atrium 204 and the right ventricle 206 to the second pad 18, or vice versa. Optionally, the second pad 18 may be moved to the side or behind the left ventricle 208. A therapy vector 210 may pass from the first pad 16 through the right atrium 204, the left atrium 212, and to the second pad 18, or vice versa. Alternatively, the first pad 16 may be moved to the side, behind, or in front of the left atrium 212, while the second pad 18 is moved to the side, behind, or in front of the right ventricle 206. With such placement, a therapy vector 214 may pass between the first and second pads 16 and 18 and through the right atrium 206 and the left ventricle 212. The pads 16 and 18 may be positioned at various locations relative to the heart 200, so that various therapy vectors, other than those shown, may pass through various portions of the heart 200, including the right and left atrium 202, 212, the right and left ventricles 206, 208, the superior vena cava 220, the inferior vena cava 222, and the like.

Referring again to FIG. 1, the ESMD 10 provides a low-profile system that may include the relatively small first and second pads 16 and 18 connected together through the coupling member 20. The ESMD 10 may be devoid of a vest, shirt, or the like that retains and positions electrodes. Instead, the ESMD 10 provides pads 16 and 18 that directly mount and adhere and adhesively secure to the skin of the patient 14. The patient-engaging surfaces, such as bottom surfaces, of the pads 16 and 18 directly engage the skin of the patient, such as through the conductive adhesive. The ESMD 10 is configured to be worn by the patient for an extended period of time, such as days, weeks, and even months. The flexible, resilient pads 16 and 18 are adhesively secured to an outer skin surface of the patient 14, thereby allowing the patient 14 to wear the ESMD 10 comfortably and over long periods of time. Moreover, the ESMD 10 may be water-proof, so that the patient 14 may wear the ESMD 10 during showers and bathing.

As noted, the ESMD 10 is flexible and configured to bend and flex in relation to the skin and anatomical features of the patient 14. The ESMD 10 adhesively secured to the patient 14, and the pads 16, 18 and the coupling member 20 conform to the shape of the skin, bone, and the like of the patient 14. As the patient 14 moves, such as by breathing, for example, the pads 16, 18 and the coupling member 20 flex, move, bend, twist, turn, and the like in response to the movement of the patient 14. The flexible, resilient ESMD 10 automatically reacts and follows the movement of the portion of the patient to which the ESMD 10 is adhesively secured. The ESMD 10 flexibly reacts to the body shape and movement of the patient 14.

FIG. 2 illustrates a simplified schematic diagram of an ESMD 10, according to an embodiment of the present disclosure. The ESMD 10 may include the first and second pads 16 and 18 connected together through the coupling member 20. The first pad 16 may include an electrode 30 operatively connected to one or more capacitors 32 through one or more switches 34. The electrode 30 may be similar to the electrode 25′ shown and described in FIG. 1 c, for example. The first pad 16 may also include a battery 35 that supplies power to the ESMD 10. The electrode 30 is configured to be positioned on the securing strip 22 so that it is able to direct electrical stimulus into the skin of the patient 14 (shown in FIG. 1). The battery 35, the capacitor 32, and the switch 34 may be contained within the housing 24 (shown in FIG. 1) of the first pad 16.

The second pad 18 may include an electrode 36 connected to one or more capacitors 38 through one or more switches 40. The electrode 36 may be similar to the electrode 25′ shown and described in FIG. 1 c, for example. The second pad 18 may also include a control unit 42 operatively connected to the electrode 36, the capacitor 38, and the switch 40. The control unit 42 may also be operatively connected to a memory 44, a telemetry circuit 46, and a speaker 48. The control unit 42, the memory 44, the telemetry circuit 46, the speaker 48, the capacitor 38 and the switch 40 may be contained within the housing 24 (shown in FIG. 1).

The capacitors 32 and 38 may be various types of flexible capacitors or charge storage elements. The capacitors 32 and 38 represent charge storage elements that are used in connection with delivering stimulation therapy. For example, the capacitors 32 and 38 may each include one or more dielectric capacitors or electrochemical capacitors, also known as supercapacitors. As a more specific example, the capacitors 32 and 38 may include one or more solid-state supercapacitors based on carbon nanoparticles (CNPs)/MnO₂ nanorods hybrid structure using a polyvinyl alcohol (PVA)/H₃PO₄ electrolyte. As another example, the capacitors 32, 38 may be laser-scribed grapheme (LSG) based capacitors. As another example, the capacitors 32, 38 may be surface mounted multi-layer ceramic capacitors having a conductive epoxy between conductive metal layers. Optionally, the capacitors 32 and 38 may represent aluminum or tantalum electrolytic capacitors.

The battery 35 may be a standard battery, or it may be a flexible battery. The battery 35 may be thin and flexible, and configured to provide power to the ESMD 10. For example, the battery 35 may include a flexible substrate including flexible sheets of polydimethylsiloxane (PDMS). The substrate may support a lithium cobalt oxide cathode, a solid electrolyte, and a lithium-metal anode.

In general, the charge storage elements (for example, the capacitors 32, 38) and the battery 35 may be thin and flexible. LSG layers may be positioned on a flexible substrate. As such, the capacitors 32, 38 and the battery 35 may flex and move along with movement of the chest or other such outer skin area of the patient.

The control unit 42 may include a programmable microcontroller that controls various operations of ESMD 10, including cardiac monitoring and stimulation therapy. The microcontroller may include a microprocessor (or equivalent control circuitry), RAM and/or ROM memory, logic and timing circuitry, state machine circuitry, and I/O circuitry. The control unit 42 is operatively connected to, and in communication with, the memory 44, which may store operating instructions for the control unit 42. The memory may include RAM and/or ROM memory, flash memory, or the like. The control unit 42 may store patient data on the memory 44, such as therapy delivery dates, times, energy levels, and the like. The control unit 42 and memory 44 may communicate with an external device through the telemetry circuit 46. As such, the control unit 42 may be programmed through an external device, and patient data may be communicated between the control unit 42 and the external device through the telemetry circuit 46.

The control unit 42 controls operation of the ESMD 10. Accordingly, the control unit 42 is operatively connected to the first pad 16 through a control line 50 within the coupling member 20. The control unit 42 is operatively connected to the capacitor 32, the switch 34 and the electrode 30 of the first pad 16 through the control line 50.

The second pad 18 may be powered through the battery 35 within the first pad 16. As such, the battery 35 may be connected to the second pad 18 through a power line 52 that allows battery power to be transferred from the battery 35 to the control unit 42, the capacitor 38, and the various other components of the second pad 18. Additionally, the coupling member 20 may also contain a therapy delivery line 53 configured to allow shocking therapy energy to be delivered to the pads 16 and 18. Optionally, the control line 50, the power line 52, and the therapy delivery line 53 may be integrated into a single line that allows power, energy, and control signals to be transferred between the first and second pads 16 and 18.

The control unit 42 is configured to operate the speaker 48 to provide audible alerts to a patient. For example, the control unit 42 may sound an audible alert to a patient that a therapeutic shock will be delivered in a certain period of time, such as five seconds. The ESMD 10 may also include a user interface having one or more buttons that allow a patient to override the impending shock. Alternatively, the ESMD 10 may not include the speaker 48 and/or the user interface.

Referring to FIGS. 1 and 2, the EMSD 10 is secured to the patient 14. For example, the EMSD 10 may be secured to the patient after the patient 14 has been treated for a heart attack. Medical adhesives are applied to the undersides of the securing strips 22 and then the first and second pads 16 and 18 are urged into desired positions on the patient 14. The medical adhesives ensure that the patient-engaging surfaces, such as the bottom surfaces, of the first and second pads 16 and 18 remain securely connected to the patient 14 without any intervening structure between the outer surface of the skin of the patient and the underside of the pads 16 and 18, except the conductive adhesive.

As discussed above, the ESMD 10 is chronically present with respect to the patient 14 in that the ESMD 10 is adhesively secured to the patient 14 over long periods, such as days, weeks, and months, whether the patient 14 experiences adverse cardiac episodes or not. Moreover, the ESMD 10 is flexible, resilient, and conforms to the shape and contours of a patient's skin, anatomical structure, and the like. Due to its flexibility and resiliency, the ESMD 10 is configured to be adhesively secured to a portion of a patient over long periods of time, such as days, weeks, and months, while continually adapting to patient movement (such as by bending, twisting, turning, etc.) caused by, for example, breathing, postures, poses, and the like.

The EMSD 10 may be particularly useful during the period of time in which the patient 14 is susceptible to an adverse arrhythmic sudden death after being treated for a heart attack, for example. The EMSD 10 is configured to continually monitor cardiac activity of the patient 14, and provide therapy, when necessary. Accordingly, the patient 14 may not need to rely on a separate and distinct device, such as a remote defibrillator, or risk surgery to have a separate and distinct device implanted within anatomy of the patient.

Failure to monitor post myocardial infarction arrhythmia may increase the risk of stroke, clots, and other arrhythmia-related conditions. The EMSD 10 continually monitors the heart function of the patient 14, and delivers therapy when appropriate. The EMSD 10 may be particularly useful during a forty day window after treatment of a myocardial infarction, in which the patient would otherwise be susceptible to another adverse cardiac episode including serious arrhythmias.

In operation, an arrhythmia may trigger the control unit 42 to deliver a shock to the heart of the patient 14. The control unit 42 may then cause one or both of the capacitors 32 and 38 to charge. At the same time, the control unit 42 may sound an audible alarm to the patient 14 through the speaker 48. The audible alarm may alert the patient that a defibrillating shock will occur in a short period of time, such as five seconds. The patient 14 may have a short period of time to abort the shock. If the patient decides to abort the shock, the patient 14 engages an abort button or user interface on one or both of the pads 16 and 18.

The control unit 42 may store episodes of adverse cardiac activity, such as atrial tachycardia, atrial fibrillation, ventricular tachycardia, ventricular fibrillation, and/or the like, in the memory 44. The data in the memory 44 may later be accessed through a standard communication interface between the memory 44 and a computing system (not shown), and/or through the telemetry circuit 46. Moreover, the telemetry circuit 46 may immediately communicate data of an adverse cardiac event to an external device.

Additionally, because the EMSD 10 is quickly and easily secured to the patient 14, the EMSD 10 may be just as easily accessed for maintenance or replacement, in stark contrast to a medical device that has been implanted within anatomy of a patient. For example, the battery 35 of the EMSD 10 may be easily accessed, removed from, and replaced in a housing of the pad 16 or 18.

The material and labor costs associated with the ESMD 10 are less than those of implantable devices. In contrast to an implantable device, the ESMD 10 may use standard, non-implantable batteries and capacitors. Additionally, the ESMD 10 may include lower cost electronics, such as standard surface mount technologies on epoxy-glass circuit boards.

The components of the ESMD 10, such as the control unit 42, the memory 44, the telemetry circuit 46, and/or the speaker 48 may be part of a flexible or flex circuit. Each of the components of the ESMD 10 may be mounted on a flexible plastic substrate, such as polymide, polyether ether ketone (PEEK), transparent conductive polyester film, or the like. The substrate may be plastic or metal foil, for example. Thus, for example, the electrode 30, the switch 34, and the capacitor 32 of the first pad 16 may be part of a flex circuit, while the electrode 36, the capacitor 38, the switch, the control unit 42, the memory 44, and the telemetry circuit 46 may also be part of one or more flex circuits.

Alternatively, each of the first and second pads 16 and 18 may include separate and distinct batteries and separate and distinct control units. Further, the battery 35 may be alternatively positioned within the second pad 18, while the control unit 42, the memory 44, the telemetry circuit 46, and the speaker 48 may be positioned within the first pad 16. Additionally, the second pad 18 may include the control unit 42, while the first pad 16 may include the memory 44, the telemetry circuit 46, and the speaker 48.

As shown in FIG. 2, each electrode 30 and 36 may be connected to a separate and distinct capacitor(s) 32 and 38, respectively. Optionally, the electrodes 30 and 36 may be operatively connected to a single capacitor or bank of capacitors.

FIG. 3 illustrates a simplified schematic diagram of first and second electrodes 60 and 62, respectively, connected to a charge storage element 64, according to an embodiment of the present disclosure. The first and second electrodes 60 and 62 may be positioned within first and second pads, respectively, such as the first and second pads 16 and 18 shown in FIGS. 1 and 2. The charge storage element 64 may be positioned within one of the first and second pads 16 or 18, and connected to each of the first and second electrodes 60 and 62 through charge delivery lines 66 and 68, respectively, that may be contained within the coupling member 20 (shown in FIGS. 1 and 2).

The charge storage element 64 may include multiple capacitors 70 connected in series. Optionally, the capacitors 70 may be connected in parallel. Each of the first and second electrodes 60 and 62 are connected to the capacitors 70 through respective switches 72 and 74. The charge storage element 64 may include more or less capacitors 70 than shown.

While shown connected to two electrodes 60 and 62, the charge storage element 64 may be connected to a single electrode, such as described with respect to FIG. 1c. As an example, four to eight capacitors may be used. The capacitors may be arranged in parallel or series. For example, the capacitors may be evenly divided into two groups, in which each group includes an equal number of capacitors in parallel or series. The groups of capacitors may also be in parallel or series. For example, one group of four capacitors may be in parallel with another group of four capacitors. The capacitors may be roll, flash, or flat capacitors, for example. In terms of size, each capacitor may have dimensions of 1 cm×3 cm×4 cm, although smaller or larger capacitors may be used.

The charge storage element 64 may be capable of delivering therapy at 80-150 Joules, for example, the charge storage element 64 may be neutral before delivery. Prior to delivery of therapy, the charge storage element 64 may charge, such as in less than one minute.

FIG. 3b illustrates a simplified configuration for a charge storage element 64′, according to an embodiment of the present disclosure. The charge storage element 64′ may include a first bank 65′ of capacitors 70′ connected to a second bank 67′ of capacitors 70′ in parallel. Each capacitor 70′ may be a 200 Farad capacitor, for example. As shown, each bank 65′ and 67′ may include four capacitors 70′ in series with one another. More or less capacitors 70′ may be included in each bank 65′ and 67′. Moreover, the capacitors 70′ in each bank may be in parallel, instead of series. Further, instead of being connected in parallel, the banks 65′ and 67′ may be connected together in series.

FIG. 4 illustrates a simplified schematic diagram of an ESMD 80, according to an embodiment of the present disclosure. The ESMD 80 may include a first pad 82 connected to a second pad 84 through a coupling member 86. Each of the first and second pads 82 and 84 may include one or more electrodes 88 operatively connected to a switch 90. The first pad 82 may include a control unit 92 operatively connected to a memory 94, a telemetry circuit 96, a speaker 98, a battery 100, and a charge storage element 102. The charge storage element 102 is operatively connected to the electrodes 88 of the first and second pads 82 and 84, and may be configured as shown and described with respect to FIG. 3.

Alternatively, the charge storage element 102 may be within the second pad 84. Also, alternatively, at least a portion of the various components shown in the first pad 82 may be positioned within the second pad 84.

FIG. 5 illustrates a simplified schematic diagram of an ESMD 500, according to an embodiment of the present disclosure. The ESMD 500 may include first and second pads 502 and 504, respectively, operatively connected by a coupling member 506. The pads 502 and 504 are similar to those described above. The first pad 502 may include a plurality of electrodes 508a, 508b, 508c, and 508d. The second pad 504 may include a plurality of electrodes 510a, 510b, 510c, and 510d. Each of the electrodes 508a-d and 510a-d may be operatively connected to a switch, which, in turn, may be connected to one or more capacitors, as described above. Further, each pad 502 and 504 may include more or less electrodes than shown.

The electrodes 508a-d and 510a-d may be positioned in different orientations. For example, the electrodes 508a and 508c may be longitudinally aligned with the first pad 502 (for example, parallel with the longitudinal axis X of the first pad 502), while the electrode 508b may be laterally aligned with the first pad 502 (for example, parallel with the lateral axis Y of the first pad 502). Further, the electrode 508d may be angled with respect to longitudinal axis x and lateral axis y of the first pad 502. Similarly, the electrodes 510a and 510c of the second pad 504 may be longitudinally aligned with the second pad 504 (for example, parallel with the longitudinal axis X of the second pad 504), while the electrode 510b may be laterally aligned with the second pad 504 (for example, parallel with the lateral axis Y of the second pad 504). Further, the electrode 510d may be angled with respect to longitudinal axis x and lateral axis y.

The electrodes 508a and 508c, as well as the electrodes 510a and 510c may be configured to be longitudinally aligned with ribs, for example, of a patient. As an example, the electrodes 508a, 508c, 510a, and 510c may be configured to overlay interstitial spaces between ribs of a patient. When the electrodes 508a, 508c, 510a, and 510c are aligned with respect to the ribs, the electrodes 508a, 508c, 510a, and 510c generally avoid overlaying portions of the ribs, thereby providing a relatively clear path towards cardiac tissue.

Similarly, the electrodes 508b and 510b may be configured to align with respect to other portions of anatomy. As example, the electrodes 508b and 510b may be oriented to avoid passing through lungs, or other anatomical structure, in order to have a relatively unimpeded path towards cardiac tissue.

Each pad 502 and 504 may include a plurality of electrodes configured in various orientations that are configured to be proximate to, but not overlay, particular portions of anatomy. As such, the various electrode configurations may be uniquely configured to be proximate to different portions of anatomy without overlaying the various portions of anatomy.

The multiple electrodes 508a-d and 510a-d are configured to provide shocking therapy along multiple vectors between the first and second pads 502 and 504. Additionally, the varying orientations of the electrodes 508a-d and 510a-d allow an operator to configure the pads 502 and 504 for a select or optimal performance. For example, the varying orientations of the electrodes 508a-d and 510a-d allow manual or automatic calibration of the ESMD 500 in order to determine a desired electrode configuration and orientation for high voltage vectors between electrodes 508a-d and 510a-d of the first and second pads 502 and 504, respectively. An individual may detect high voltage vectors between pairs of the electrodes 508a-d and 510a-d. As an example, a high voltage vector between the electrode 508d and the electrode 510a may be more clearly correlated with an electrocardiography (EKG signal than between other pairs of electrodes. Accordingly, after the ESMD 500 is secured to a patient, medical personnel may calibrate the ESMD 500 by determining which pairs of electrodes 508a-d and 510a-d provide clear and efficient monitoring and therapy.

FIG. 6 illustrates a simplified schematic diagram of an ESMD 600, according to an embodiment of the present disclosure. The ESMD 600 includes first and second pads 602 and 604 operatively connected together through a first coupling member 606. Additionally, a third pad 608 is connected to the second pad through a second coupling member 610. Each of the pads 602, 604, and 608 may be configured similar to any of the pads discussed above. The additional pad 608 allows the ESMD 600 to provide monitoring and therapy over extended distances. The ESMD 600 may include additional pads and coupling members.

As shown in FIG. 6, each pad 602, 604, and 608 may include two parallel electrodes 620. Within each pad 602, 604, and 608, the electrodes 620 may be separated by a gap, which may be configured to overlay a rib of a patient. The electrodes 620 may therefore be configured to be in a straddling relationship with respect to the rib, and overlay interstitial spaces between ribs. However, each pad 602, 604, and 608 may include alternative electrode configurations, such as discussed above.

FIG. 7 illustrates a simplified schematic diagram of an ESMD 700, according to an embodiment of the present disclosure. The ESMD 700 includes a first pad 702 connected to a second pad 704 through a first coupling member 706. A third pad 708 is, in turn, connected to the second pad 704 through a second coupling member 710. Further, a fourth pad 712 is, in turn, connected to the third pad 708 through a third coupling member 714. Therefore, the ESMD 700 provides an extended system that allows the ESMD 700 to extend over longer distances of patient anatomy and monitor cardiac activity (and provide therapy) at more locations. Each pad 702, 704, 708, and 712 may be configured similar to any of the pads described above.

As shown in FIG. 7, each pad 702, 704, 708, and 712 may include two parallel electrodes 720. Within each pad 702, 704, 708, and 712 the electrodes 720 may be separated by a gap, which may be configured to overlay a rib of a patient. The electrodes 720 may therefore be configured to be in a straddling relationship with respect to the rib, and overlay interstitial spaces between ribs. However, each pad 702, 704, 708, and 712 may include alternative electrode configurations, such as discussed above.

FIG. 8 illustrates a top view of a pad 800 of an ESMD, according to an embodiment of the present disclosure. The pad 800 may include positioning indicia 802 and 804 of electrode locations within the pad 800. The pad 800 may also include positioning indicia 806 with respect to anatomical features. For example, the positioning indicia 802 and 804 may be shapes that mirror the shape of the underlying electrode, while the positioning indicia may be sized and shaped as a rib. Alternatively, the positioning indicia 802 and 804 may merely be generic shapes, such as blocks, curves, or the like, and/or text, such as “Electrode,” “Rib,” and/or the like. As shown in FIG. 8, the pad 800 may include positioning indicia 806 that indicate where the pad 800 should be positioned with respect to a rib of a patient. The positioning indicia 806 is configured to be aligned along and over a rib, for example, while the positioning indicia 804 and 806 are configured to be aligned along and overlay interstitial spaces or gaps on either side of the rib. Thus, the electrodes underlying the positioning indicia 802 and 804 are to be positioned over the interstitial spaces or gaps on opposite sides of the rib. In this manner, the positioning indicia 802, 804, and 806 allow an individual to position the pad 800 on a patient so that the path between the electrodes and the heart, for example, is not blocked by ribs, for example. As such, electrical signals from the electrodes are not attenuated or otherwise absorbed by the ribs, for example.

More or less positioning indicia than shown may be used. The positioning indicia are used to allow for optimal electrode placement on the patient. The positioning indicia are used so that an individual may place the pad 800 in order to minimize or otherwise reduce attenuation of the electrical signals from bone structure, such as ribs. Any of the pads described above may include the positioning indicia.

Referring to FIG. 1-8 above, each of the pads may include one or more lights, such as light emitting diodes (LEDs). The lights may be used to assist in proper positioning of the pads. For example, when the pads are improperly positioned, a red light may be emitted. When the pads are in proper position, the red light may switch to a green light, alerting an individual that the pad has been properly positioned. Optionally, instead of, or in addition to, lights, one or both pads may include a small speaker that emits audible cues related to improper and proper positioning. Improper and proper positioning may be based on a detected EKG signal or impedance check in relation to one or more electrodes within the pads.

FIG. 9 illustrates an exemplary block diagram of the electrical components of an ESMD 901, according to an embodiment of the present disclosure. The ESMD 901 may be implemented as a full-function biventricular pacemaker, equipped with both atrial and ventricular sensing and pacing circuitry for four chamber sensing and stimulation therapy (including both pacing and shock treatment). Optionally, the ESMD 901 may provide full-function cardiac resynchronization therapy. Alternatively, the ESMD 901 may be implemented with a reduced set of functions and components. For instance, the ESMD 901 may be implemented without ventricular sensing and pacing.

The ESMD 901 includes one or more housings 900, such as within one or more pads, as described above, that securely contain the electronic/computing components. The housing 900 may be programmably selected to act as the return electrode for certain stimulus modes. The housing 900 further includes a connector (not shown) with a plurality of terminals 903, 904, 906, 908, and 910. The terminals may be connected to electrodes of one or more pads, as shown in FIGS. 1, 2, and 4-8. For example, the terminals may include: a terminal 903 to be coupled to a first electrode located on the skin above a first chamber; a terminal 904 to be coupled to a second electrode located on the skin above a second chamber; a terminal 906 to be coupled to an electrode located on the skin above the first chamber; a terminal 908 to be coupled to an electrode located on the skin above the second chamber; and a terminal 910 to be coupled to an electrode located on the skin above the superior vena cava. The type and location of each electrode may vary. For example, the electrodes may include various combinations of ring, tip, coil and shocking electrodes and the like.

The ESMD 901 includes a programmable microcontroller 920 that controls various operations of the ESMD 901, including cardiac monitoring and stimulation therapy. Microcontroller 920 includes a microprocessor (or equivalent control circuitry), RAM and/or ROM memory, logic and timing circuitry, state machine circuitry, and I/O circuitry.

ESMD 901 further includes a first chamber pulse generator 922 that generates stimulation pulses for delivery by one or more electrodes coupled thereto. The pulse generator 922 is controlled by the microcontroller 920 via control signal 924. The pulse generator 922 is coupled to the select electrode(s) via an electrode configuration switch 926, which includes multiple switches for connecting the desired electrodes to the appropriate I/O circuits, thereby facilitating electrode programmability. The switch 926 is controlled by a control signal 928 from the microcontroller 920.

In the example of FIG. 9, a single pulse generator 922 is illustrated. Optionally, the ESMD 901 may include multiple pulse generators, similar to pulse generator 922, where each pulse generator is coupled to one or more electrodes and controlled by the microcontroller 920 to deliver select stimulus pulse(s) to the corresponding one or more electrodes.

Microcontroller 920 is illustrated as including timing control circuitry 932 to control the timing of the paceing pulses. Pacing is typically performed immediately after delivering a defibrillation pulse when there is often a period of asystole that is treated with post shock pacing. The timing control circuitry 932 may also be used for the timing of refractory periods, blanking intervals, noise detection windows, evoked response windows, alert intervals, marker channel timing, and so on. Microcontroller 920 may also include an arrhythmia detector 934 for detecting arrhythmia conditions and a morphology detector 936. Although not shown, the microcontroller 920 may further include other dedicated circuitry and/or firmware/software components that assist in monitoring various conditions of the patient's heart and managing pacing therapies.

The ESMD 901 may be further equipped with a communication modem (modulator/demodulator) 940 to enable wireless communication. In one implementation, the communication modem 940 uses high frequency modulation. As one example, the modem 940 transmits signals between a pair of electrodes of the lead assembly, such as between the housing(s) 900 and the right ventricular tip electrode. The signals are transmitted in a high frequency range of approximately 20-80 kHz, as such signals travel through the body tissue in fluids without stimulating the heart or being felt by the patient.

The communication modem 940 may be implemented in hardware as part of the microcontroller 920, or as software/firmware instructions programmed into and executed by the microcontroller 920. Alternatively, the modem 940 may reside separately from the microcontroller as a standalone component.

The ESMD 901 may include a sensing module 944 selectively coupled to one or more electrodes that perform sensing operations, through the switch 926 to detect the presence of cardiac activity in the corresponding chambers of the heart. The sensing module 944 is configured to sense cardiac activity and detect adverse cardiac events, such as arrhythmias. The sensing module 944 may be configured to perform bipolar sensing between one pair of electrodes and/or between multiple pairs of electrodes. The sensing module 944 may include dedicated sense amplifiers, multiplexed amplifiers, or shared amplifiers. The sensing module 944 may further employ one or more low power, precision amplifiers with programmable gain and/or automatic gain control, bandpass filtering, and threshold detection circuit to selectively sense the cardiac signal of interest. The automatic gain control enables the unit to sense low amplitude signal characteristics of atrial fibrillation. Switch 926 determines the sensing polarity of the cardiac signal by selectively closing the appropriate switches. In this way, the clinician may program the sensing polarity independent of the stimulation polarity.

The output of the sensing module 944 is connected to the microcontroller 920 which, in turn, triggers or inhibits the pulse generator 922 in response to the absence or presence of cardiac activity. The sensing module 944 receives a control signal 946 from the microcontroller 920 for purposes of controlling the gain, threshold, polarization charge removal circuitry (not shown), and the timing of any blocking circuitry (not shown) coupled to the inputs of the sensing circuitry.

In the example of FIG. 9, a single sensing module 944 is illustrated. Optionally, the ESMD 901 may include multiple sensing modules, similar to sensing module 944, where each sensing module is coupled to one or more electrodes and controlled by the microcontroller 920 to sense electrical activity detected at the corresponding one or more electrodes. The sensing module 944 may operate in a unipolar sensing configuration or in a bipolar sensing configuration.

The ESMD 901 may also include an analog-to-digital (ND) data acquisition system (DAS) 950 coupled to one or more electrodes via the switch 926 to sample cardiac signals across any pair of desired electrodes. The data acquisition system 950 is configured to acquire intracardiac electrogram signals, convert the raw analog data into digital data, and store the digital data for later processing and/or telemetric transmission to an external device 954 (e.g., a programmer, local transceiver, or a diagnostic system analyzer). The data acquisition system 950 is controlled by a control signal 956 from the microcontroller 920.

The microcontroller 920 is coupled to a memory 960 by a suitable data/address bus 962. The programmable operating parameters used by the microcontroller 920 are stored in memory 960 and used to customize the operation of the ESMD 901 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart within each respective tier of therapy.

The operating parameters of the ESMD 901 may be programmed into the memory 960 through a telemetry circuit 964 in telemetric communication via communication link 966 with the external device 954. The telemetry circuit 964 allows intracardiac electrograms and status information relating to the operation of the ESMD 901 (as contained in the microcontroller 920 or memory 960) to be sent to the external device 954 through the established communication link 966.

The ESMD 901 may also include magnet detection circuitry (not shown), coupled to the microcontroller 920, to detect when a magnet is placed over the unit. A magnet may be used by a clinician to perform various test functions of the unit 902 and/or to signal the microcontroller 920 that the external programmer 954 is in place to receive or transmit data to the microcontroller 920 through the telemetry circuit 964.

The ESMD 901 may also include one or more physiologic sensors 970. Such sensors are commonly referred to as “rate-responsive” sensors because they are typically used to adjust pacing stimulation rates according to the exercise state of the patient. However, the physiological sensor 970 may further be used to detect changes in cardiac output, changes in the physiological condition of the heart, or diurnal changes in activity (e.g., detecting sleep and wake states). Signals generated by the physiological sensors 970 are passed to the microcontroller 920 for analysis. While shown as being included within the unit 902, the physiologic sensor(s) 970 may be external to the ESMD 901, yet still be implanted within or carried by the patient. Examples of physiologic sensors might include sensors that, for example, sense respiration rate, ventricular gradient, activity, position/posture, minute ventilation (MV), SpO2, and so forth. Information from these sensors may be analyzed and stored so as to provide a profile of the patient's condition.

A battery 972 provides operating power to all of the components in the ESMD 901. The battery 972 is capable of operating at low current drains for long periods of time, and is capable of providing high-current pulses (for capacitor charging) when the patient requires a shock pulse (e.g., in excess of 2 A, at voltages above 2 V, for periods of 10 seconds or more). The battery 972 also desirably has a predictable discharge characteristic so that elective replacement time can be detected. As one example, the ESMD 901 employs lithium/silver vanadium oxide batteries.

The ESMD 901 may also include an impedance measuring circuit 974, which can be used for many things, including: lead impedance surveillance for detecting detachment; detecting operable electrodes and automatically switching to an operable pair if detachment occurs; measuring respiration or minute ventilation; measuring thoracic impedance for determining shock thresholds; measuring stroke volume using impedance cardiography; and detecting the opening of heart valves; and so forth from the impedance cardiographic signals as is known in the art. The impedance measuring circuit 974 is coupled to the switch 926 so that any desired electrode may be used.

The microcontroller 920 further controls a shocking circuit 980 by way of a control signal 982. The shocking circuit 980 generates shocking pulses of low (e.g., up to 0.5 joules), moderate (e.g., 0.5-40 joules), or high energy (e.g., 40 to 200 joules), as controlled by the microcontroller 920. The shocking circuit 980 may include one or more charge storage elements 981 that are configured to deliver stimulation therapy to an internal organ, such as a heart, of a patient.

Optionally, after defibrillation or cardioversion, when shocks are performed, the heart may go asystolic for a period of several seconds. Therefore, low energy stimulation in the form of cardiac pacing is typically performed with pulses of 10 to 50 milliseconds in duration using currents from 10 to 200 milliamperes at rates of 60 to 120 pulses per minute. These pacing pulses may be delivered in a transcutaneous VVI demand mode as is well known in cardiac pacing. The ECG may be used to determine if a spontaneous depolarization has occurred to obviate the need to pace. Hemodyanamic efficacy of pacing may be verified using impedance or photoplethysmography. Pacing may also be performed without a preceding defibrillation shock. Pacing need is indicated by the absences of a cardiac depolarization or prolonged intervals. Pacing may also be performed using rate hysteresis.

FIG. 10 illustrates a functional block diagram of an external device 1000 that is operated in accordance with the processes described herein and to interface with ESMDs, according to an embodiment of the present disclosure. The external device 1000 may be a workstation, a portable computer, an IMD programmer, a PDA, a cell phone, or the like. The external device 1000 includes an internal bus that connects/interfaces with a Central Processing Unit (CPU) 1002, ROM 1004, RAM 1006, a hard drive 1008, a speaker 1010, a printer 1012, a CD-ROM drive 1014, a floppy drive 1016, a parallel I/O circuit 1018, a serial I/O circuit 1020, a display 1022, a touch screen 1024, a standard keyboard connection 1026, custom keys 1028, and a telemetry subsystem 1030. The internal bus may be an address/data bus that transfers information between the various components described herein. The hard drive 1008 may store operational programs as well as data, such as waveform templates and detection thresholds.

The CPU 1002 may include a microprocessor, a micro-controller, or equivalent control circuitry, designed specifically to control interfacing with the external device 1000 and with an ESMD. The CPU 1002 may include RAM or ROM memory, logic and timing circuitry, state machine circuitry, and I/O circuitry to interface with the ESMD. The touch screen 1024 may display graphic information 1032 relating to the ESMD. The display 1022 displays various information related to the processes described herein. The touch screen 1024 accepts a user's touch input 1034 when selections are made. The keyboard 1026 (e.g., a typewriter keyboard) allows the user to enter data to the displayed fields, as well as interface with the telemetry subsystem 1030. Furthermore, custom keys 1028 turn on/off 1038 (e.g., EVVI) the external device 1000. The printer 1012 prints copies of reports 1040 for a physician to review or to be placed in a patient file, and speaker 1010 provides an audible warning (e.g., sounds and tones 1042) to the user. The parallel I/O circuit 1018 interfaces with a parallel port 1044. The serial I/O circuit 1020 interfaces with a serial port 1046. The floppy drive 1016 accepts diskettes 1048. Optionally, the floppy drive 1016 may include a USB port or other interface capable of communicating with a USB device such as a memory stick. The CD-ROM drive 1014 accepts CD ROMs 1050.

The telemetry subsystem 1030 includes a central processing unit (CPU) 1052 in electrical communication with a telemetry circuit 1054, which may communicate with both an ECG circuit 1056 and an analog out circuit 1058. The circuit 1056 may be connected to leads 1060. The circuit 1056 is also connected to the leads to receive and process cardiac signals as discussed above. Optionally, the cardiac signals sensed by the electrodes may be collected by the ESMD and then transmitted, to the external device 1000, wirelessly to the telemetry subsystem 1030 input.

The telemetry circuit 1054 may be connected to a telemetry wand 1062. The analog out circuit 1058 includes communication circuits to communicate with analog outputs 1064. The external device 1000 may wirelessly communicate with the ESMD and utilize protocols, such as Bluetooth, GSM, infrared wireless LANs, HIPERLAN, 3G, satellite, as well as circuit and packet data protocols, and the like. Alternatively, a hard-wired connection may be used to connect the external device 1000 to the ESMD.

FIG. 11 illustrates a flow chart of a process of securing an ESMD to a patient, according to an embodiment of the present disclosure. At 1100, adhesive is applied to an underside of one or more pads of the ESMD. For example, if the ESMD includes first and second pads, the adhesive is applied to undersides of both pads. If the adhesive is an integral part of the pad, it may be necessary to peel off a protected cover 31 that is placed over the adhesive to expose the pad as shown in FIG. 1 b.

Next, at 1102, proper positions of the pad(s) are determined with respect to skin of the patient. Each pad may include positioning indicia that allow a clinician to determine the locations of electrodes within the pads. The clinician may utilize the positioning indicia to ensure that the electrodes do not overlay bone structure, such as ribs. As an example, the pads may be positioned such that one or more electrodes are aligned along and overlay interstitial spaces or gaps between ribs.

Once the proper positioning for the pad(s) is determined, the pad(s) are pressed into the skin at the proper locations at 1104. When pressed into the skin, the adhesives securely and safely connect the pad(s) to the skin. Then, at 1106, the patient may simply put a shirt over his/her chest, thereby covering the ESMD. In this manner, the ESMD provides a simple, discrete, and compact device that may secured to the skin, without requiring the patient to wear any specific or specially-designed clothing that integrates the ESMD. The ESMD is configured to be worn by the patient for an extended period of time, such as days, weeks, and even months.

Additionally, the method may include, at 1108, detecting a baseline EKG after the ESMD has been secured to the patient. The baseline EKG is used to determine an arrhythmia. For example, the ECG may be detected in the patient during normal cardiac activity. The ECG during normal cardiac activity may be the baseline ECG. At 1110, the baseline IEGM is continually compared against current cardiac activity in a patient. When current cardiac activity varies, at least to a significant degree, with the baseline IEGM, the ESMD may determine that an arrhythmia exists, and may then deliver therapy, such as a defibrillating shock. Thus, at 1112, the ESMD determines whether the baseline ECG or EKG differs from current cardiac activity. If there is no significant difference, the process returns to 1110. If, however, there is a significant difference, the ESMD delivers therapy at 1114, and the process returns to 1110.

Embodiments of the present disclosure provide ESMDs that may be quickly and easily secured to a patient. The ESMDs continually monitor the cardiac activity of patients, without reliance on bulky systems and methods (such as remote defibrillation systems and methods) that are typically used only after a patient experiences an adverse cardiac episode. Further, the ESMDs are secured externally to a patient, over the skin, for example, but are not surgically implanted within the patient. Further, the ESMDs ensure that the patient will return to a medical facility for follow-up examination, thereby improving patient outcomes and reducing costs from complications.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the disclosure without departing from its scope. While the dimensions, types of materials and coatings described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

What is claimed is:

1. An externally-secured medical device (ESMD) configured to be securely affixed to skin of a patient, the ESMD comprising:
a sensing module configured to sense cardiac activity and detect arrhythmia;
at least one pad configured to be directly secured to the skin of the patient, the at least one pad comprising at least one charge storage element operatively connected to at least one switch, a battery configured to provide power to the ESMD and to charge the at least one charge storage element, at least one electrode configured to deliver stimulation therapy to the skin of the patient along at least one vector through cardiac tissue, and at least one control unit operatively connected to the at least one charge storage element, the switch, the battery, and the at least one electrode, wherein the control unit is configured to control operation of the ESMD to deliver the stimulation therapy when the arrhythmia is detected;
an adhesion component provided on a patient-engaging surface of the at least one pad configured to securely affix the at least one pad in a persistent and enduring manner to the skin of the patient; and
wherein the at least one pad and the at least one electrode are directly secured to the skin of the patient through the adhesion component.

2. The ESMD of claim 1, wherein the at least one pad comprises first and second pads connected together through a coupling member, and wherein the at least one electrode comprises a first electrode on the first pad, and a second electrode on the second pad.

3. The ESMD of claim 1, wherein the at least one charge storage element comprises at least one capacitor operatively connected to the at least one electrode through the at least one switch.

4. The ESMD of claim 1, wherein the at least one charge storage element comprises one or both of a dielectric capacitor or an electrochemical capacitor.

5. The ESMD of claim 1, wherein the at least one charge storage element comprises at least one flexible capacitor.

6. The ESMD of claim 1, wherein the control unit is formed on a flexible circuit.

7. The ESMD of claim 1, further comprising a memory operatively connected to the control unit.

8. The ESMD of claim 1, further comprising a telemetry circuit configured to communicate with an external device.

9. The ESMD of claim 1, further comprising a speaker configured to emit audible alerts.

10. The ESMD of claim 1, wherein the battery comprises a flexible substrate including flexible sheets of polydimethylsiloxane (PDMS).

11. The ESMD of claim 10, wherein the battery further comprises a lithium cobalt oxide cathode, a solid electrolyte, and a lithium-metal anode supported by the flexible substrate.

12. The ESMD of claim 1, wherein the at least one electrode comprises a plurality of electrodes at different orientations.

13. The ESMD of claim 1, wherein the at least one pad comprises three or more pads connected together through a plurality of coupling members.

14. The ESMD of claim 1, wherein the at least one pad comprises positioning indicia configured to allow a clinician to properly position the at least one pad on the skin of the patient.

15. A method of securing an externally-secured medical device (ESMD) to skin of a patient, the method comprising:
applying an adhesion component to at least one pad of the ESMD;
determining a position of the at least one pad on the skin of the patient; and
pressing the at least one pad into the skin so that the adhesion component secures the at least one pad to the skin, wherein the pressing operation comprises positioning the at least one pad so that at least one electrode is configured to deliver stimulation therapy to the skin of the patient along at least one vector through cardiac tissue, wherein the pressing operation securely affixes the at least one pad to the skin of the patient in a persistent and enduring manner to the skin of the patient.

17. The method of claim 16, wherein the applying operation comprises applying the adhesion component to first and second pads of the ESMD, wherein the determining operation comprises determining the positions of the first and second pads on the skin of the patient, and wherein the pressing operation comprising pressing the first and second pads into the skin.

18. The method of claim 16, further comprising connecting the at least one electrode to at least one charge storage element through at least one switch.

19. The method of claim 18, wherein the at least one charge storage element comprises one or both of a flexible dielectric capacitor or a flexible electrochemical capacitor.

20. The method of claim 16, further comprising disposing a control unit configured to control operation of the ESMD within the at least one pad.
